(19) United States
(12) Patent Application Publication
(10) Pub. No.: US 2011/0178395 A1
(43) Pub. Date: Jul. 21, 2011

(54) IMAGING METHOD AND SYSTEM

(75) Inventors: Hans-Joachim Miesner, Aalen (DE); Christoph Hauger, Aalen (DE); Werner Nahm, Buehlerzell (DE); Frank Rudolph, Aalen (DE); Guido Hattendorf, Phoenix, AZ (US); Peter M. Delaney, Carnegie (AU); John D. Allen, Essendon (AU)

(73) Assignees: Carl Zeiss Surgical GmbH, Oberkochen (DE); Optiscan Pty Ltd., Notting Hill (AU)

(21) Appl. No.: 12/756,765

(22) Filed: Apr. 8, 2010

Related U.S. Application Data
(60) Provisional application No. 61/212,282, filed on Apr. 8, 2009.

(51) Int. Cl. A61B 6/00

(52) U.S. Cl. 600/425

(57) ABSTRACT

Imaging systems and methods are provided herein. An imaging system for imaging a surgical site, may include a macroscopic visualization system; and an imaging apparatus with a probe, the imaging apparatus being adapted to image the observational field and generate second image data; wherein the system is operable to control the macroscopic visualization system and the imaging apparatus to image the site and the observational field respectively at substantially the same time, and to associate the first image data and the second image data. Imaging methods provided herein may include the steps of: imaging the site with a macroscopic visualization system and generating first image data; imaging at substantially the same time an observational field with an imaging apparatus and generating second image data; and associating the first image data and the second image data.

### CROSS REFERENCE TO RELATED APPLICATIONS

This application claims the benefit of U.S. Provisional Patent Application No. 61/212,282 filed Apr. 8, 2009, the entire contents of which is hereby incorporated herein by this reference.

### FIELD OF THE INVENTION

The present invention relates generally to an imaging method and system, of particular but by no means exclusive application in endomicroscopy and in microsurgical and other procedures performed under optical stereoscopic magnified visualization, including neurosurgery, ENT/facial surgery and spinal surgery.

### BACKGROUND OF THE INVENTION

One existing microscopic probe comprises an endoscope or endomicroscope, with an endoscopic head for insertion into a patient (through the mouth or anus) coupled to a laser source by an optical fibre or optical fibre bundle. Another microscopic probe is similar to this endoscope, but adapted for examining the skin.

### SUMMARY OF THE INVENTION

According to a first aspect of the invention, therefore, there is provided an imaging system for imaging a surgical site, comprising: a macroscopic visualization system (such as an operating microscope) for imaging the site and generating first image data; and an imaging apparatus with a probe for locating at the site to define an observational field, the microscopic imaging system being adapted to image the observational field (such as a portion of or beneath the site) and generate second image data; wherein the microscopic imaging system is operable to control the macroscopic visualization system and the microscopic imaging system to image the site and the observational field respectively at substantially the same time, and to associate the first image data and the second image data. The probe is generally a manually manipulable probe.

Thus, the first image data and the second image data are associated, so that the position of the probe will be apparent in the associated image from the macroscopic visualization system. If the observational field is on or just beneath the surface of the site, the observational field—or the surface immediately above the observational field—will be visible in the associated image from the macroscopic visualization system.

The imaging apparatus may be a microscopic imaging apparatus, that is, have a higher magnification or a higher image resolution than the macroscopic visualization system. for example, an image collected with an operating microscope has a typical resolution of approximately 10 μm, whereas an image collected with an endomicroscope (as an example of an imaging apparatus) has a typical resolution of approximately 1 μm.

As will be understood by those in the art, an operating microscope is the main visualization tool of a microsurgeon. It provides high magnification of tissue and thus allows very fine surgical procedures to be performed, though does not achieve cellular or subcellular resolution. An operating microscope is typically a direct viewing binocular device with a continuous passive optical path from tissue to observer. Thus, while an operating microscope is commonly referred to as a ‘microscope’, it should not be confused with an endomicroscope, which is a specific type of microscope that typically operates with at least an order of magnitude higher magnification than an operating microscope.

The first image data may be 3D image data, indicative of a stereo image collected with the macroscopic visualization system.

The system may include a user operable image collection control adapted to prompt the system to control the macroscopic visualization system and the imaging apparatus to image the site and the observational field respectively.

The system may include a data store adapted to receive the first image data and the second image. The data store may be adapted to associate the first image data and the second image. The system may include a data output for outputting the first image data and the second image data. The system may be configured to tag the first image data with first imaging time data indicative of a time at which the macroscopic visualization system imaged the site, and to tag the second image data with second imaging time data indicative of a time at which the imaging apparatus imaged the observational field. Thus, the first imaging time data and the second imaging time data associates images made at substantially the same time.

The system may be configured to generate a single data file comprising the first image data and the second image data. Thus, the first image data and the second image data can be associated by being stored in a single data file. The system may be configured to output the first image data and the second image data at substantially the same time or sequentially. Thus, the first image data and the second image data can be associated on the basis of being outputted at substantially the same time or sequentially.

The system may comprise a navigation system, controllable to output a position of the probe. The navigation system may be configured to output the position of the probe when the imaging apparatus is controlled to image the observational field. The probe may comprise a tip, the tip being adapted to be located at the site to define an observational field, and to collect a return signal. The probe may be an endoscopic probe, such as a confocal endoscopic probe. The probe may be, for example, a neurological probe, an ENT probe, an ultrasound probe, an OCT probe or a CARS probe. The probe may have an orientation marking.

The probe may have a manually manipulable proximal portion and a straight distal portion with a distal tip for locating at a site to define an observational field and collect a return signal therefrom, wherein the straight portion has a length of between 75 mm to 205 mm, and the probe has a working length of between 125 mm to 300 mm. The probe may have a curved portion between the proximal portion and the distal portion, the curved portion providing an angle between the proximal portion and the distal portion of between 120° and 150° (more preferably between 130° and 140° and in a preferred embodiment approximately 135°).

The system may comprise a navigation system, such as a surgical navigation system, or be adapted to operate in combination with such a system. The imaging apparatus may comprise an endomicroscope.

According to a second aspect of the invention, there is provided an imaging method for imaging a surgical site, comprising: imaging the site with a macroscopic visualization system (such as an operating microscope) and generating first image data; imaging at substantially the same time an observational field with an imaging apparatus and generating second image data, the imaging apparatus having a probe for defining the observational field to be imaged thereby; and associating the first image data and the second image data.

The method may include controlling an imaging system comprising the macroscopic visualization system and the imaging apparatus to image the site and the observational field respectively at substantially the same time.

According to a third broad aspect, the invention provides an imaging system for imaging a surgical site, comprising: an imaging apparatus with a probe for locating at a site to define an observational field, the imaging apparatus being adapted to make an image of the observational field and generate image data indicative thereof; and a locating mechanism for locating the probe and generating location data indicative thereof; wherein the system is operable to control the imaging apparatus to make an image of the observational field and the locating mechanism to locate the probe at substantially the same time, and to associate the image data and the location data.

The locating mechanism may comprise a macroscopic visualization system for making an image of the site and generating site image data indicative thereof, wherein the location data comprises the site image data. The locating mechanism may comprise a navigation system for locating the probe and generating the location data.

The system may further comprise a macroscopic visualization system for making an image of the site and generating site image data indicative thereof, and the system is operable to control the macroscopic visualization system to make an image of the site, the imaging apparatus to make an image of the observational field and the navigation system to locate the probe at substantially the same time, the system being configured to associate the site image data, the image data and the location data. The navigation system may be operable to locate the macroscopic visualization system or a field of view thereof and generate location data indicative of a location of the macroscopic visualization system or the field of view.

According to a fourth broad aspect, the invention provides an imaging system for imaging a surgical site, comprising: a macroscopic visualization system for viewing the site and making an image of the site; an imaging apparatus with a probe for locating at the site to define an observational field, the imaging apparatus being adapted to image the observational field and generate image data; and a navigation system for tracking a location of the macroscopic visualization system and a location of the probe and generating respective location data indicative thereof; wherein the system is operable to control the imaging apparatus to make an image of the observational field and the navigation system to locate the macroscopic visualization system and the probe at substantially the same time, the system being configured to associate the image data and the location data.

The system may be operable to use the location data to indicate the probe location in a field of view of the macroscopic visualization system or in an image made with the macroscopic visualization system.

### BRIEF DESCRIPTION OF THE DRAWINGS

In order that the invention may be more clearly ascertained, preferred embodiments will now be described, by way of example only, with reference to the accompanying drawing, in which:

FIG. 1 is a schematic view of a surgical imaging system according to an embodiment of the present invention;

FIG. 2 is a schematic view of the confocal endomicroscopic apparatus of the system of FIG. 1;

FIG. 3 is a schematic view of the probe of the apparatus of FIG. 2;

FIG. 4 is a FIG. 4 is a schematic, perspective view of the probe of the apparatus of FIG. 2;

FIG. 5 is a schematic view of the system of FIG. 1 in use;

FIG. 6 is an exemplary pair of associated images collected with the system of FIG. 1; and

FIG. 7 is a schematic, perspective view of the probe of a surgical imaging system according to another embodiment of the present invention.

### DETAILED DESCRIPTION OF THE EMBODIMENTS

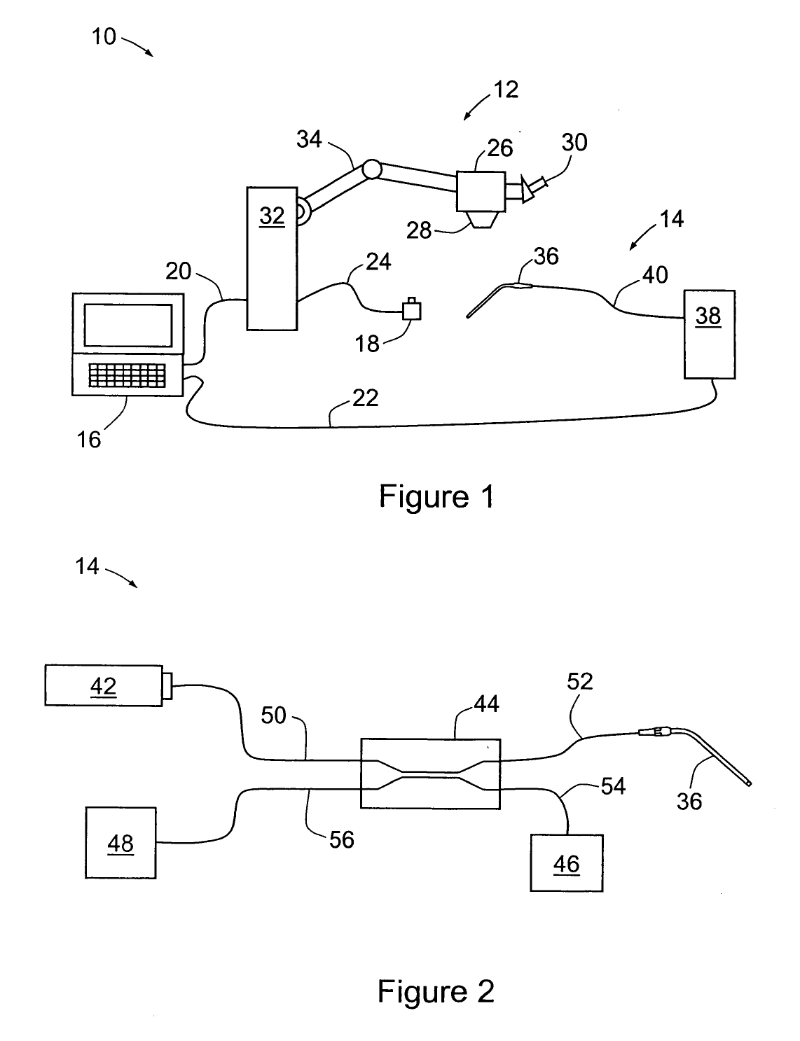

FIG. 1 is a schematic view of a surgical imaging system 10 according to an embodiment of the present invention. System 10 includes a macroscopic visualization system in the form of an operating microscope 12 for viewing and imaging a surgical site (typically accessed via a surgical access corridor created by a microsurgeon) and an imaging apparatus in the form of a confocal endomicroscope 14 for imaging at a higher magnification an observational field comprising a portion of or just beneath the site. System 10 also includes a computer 16 for controlling some of the operational parameters of operating microscope 12 and confocal endomicroscope 14, and for receiving, storing and associating image data transmitted from operating microscope 12 and confocal endomicroscope 14. System 10 includes a shutter release in the form of a footswitch 18 that, when activated by the operator (typically the microsurgeon), controls system 10 to control operating microscope 12 and confocal endomicroscope 14 to collect respective images essentially simultaneously, output image data indicative of those images, and transmit the image data to computer 16.

System 10 includes electrical cables 20, 22, 24 connecting respectively operating microscope 12 to computer 16, confocal endomicroscope 14 to computer 16, and footswitch 18 to operating microscope 12.

Computer 16 is configured to respond to the receipt of image data from either operating microscope 12 or confocal endomicroscope 14 by saving that data in its memory (not shown) with data indicative of the date and time of its creation, and of its source (either operating microscope 12 or confocal endomicroscope 14). The data indicative of the date and time of its creation associate an image from operating microscope 12 with an image collected at essentially the same time from confocal endomicroscope 14.

In an alternative embodiment, computer 16 is configured to respond to the 15 receipt of image data from operating microscope 12 and confocal endomicroscope 14 at essentially the same time by saving that data in a single data file (since it will relate to images collected at essentially the same time), with data indicative of the date and time of its creation or receipt. The file thus associates the image from operating microscope 12 with the image from confocal endomicroscope 14.

Computer 16 is also configured to respond to a command entered with its keyboard and/or mouse to display images associated in either way, so that the operator can view the site as imaged by operating microscope 12 and an observational field imaged by confocal endomicroscope 14, and determine from the image collected with operating microscope 12 which portion of the site was in the field of view of confocal endomicroscope 14 when its image was collected.

Operating microscope 12 provides a highly magnified but wide-field view of the site, and comprises an optical head 26 for viewing and collecting images of the site, with an objective housing 28 (enclosing the principal focussing optics) and binocular eyepiece 30. Optical head 26 provides a continuous passive optical path from the site to the operator. Operating microscope 12 includes a control unit 32 that houses a power supply and data processor (not shown), the latter for receiving data from a CCD (also housed in optical head 26) and forwarding that data to computer 16. Control unit 32, in addition, is configured to respond to a signal received from footswitch 18 to control operating microscope 12 to collect an image, and to control confocal endomicroscope 14—via computer 16 to which both operating microscope 12 and confocal endomicroscope 14 are connected—to collect an image at essentially the same time.

Operating microscope 12 also includes an articulated arm 34 that is supported by control unit 32 and that supports optical head 26. Confocal endomicroscope 14 comprises a confocal probe 36 and a control unit 38, coupled by a composite cable 40 that includes both an optical fibre (for transmitting excitation light to probe 36 and return light from probe 36) and electrical cable for providing power to an x-y scanning mechanism and a z scanning mechanism within probe 36. Control unit 38 includes a laser source, photodetector, light separator and other components of confocal endomicroscope 14, as are described in further detail below.

FIG. 2 is a schematic view of confocal endomicroscope 14. Confocal endomicroscope 14 includes a laser source 42 with 488 nm wavelength output, a light separator in the form of an optical coupler 44, probe 36, a power monitor 46 and a detection unit 48 in the form of a photomultiplier tube. Probe 36 includes, as mentioned above, an x-y scanning mechanism (not shown) so that light emitted by probe 36 has a point observational field that is scanned in a raster scan so that an image of the observational field can be collected and displayed. Probe 36 also includes a z scanning mechanism (not shown) so that the depth of the point observational field can be controlled. Confocal endomicroscope 14 therefore also includes electrical cables for transmitting an x-y scanning signal from control unit 38 to probe 36, for powering the scanning mechanism, and a depth control signal to a z scanning mechanism. The x-y scanning signal operates continuously when an image or images are being collected, to built up raster scans of an extended observational field.

The depth control signal instructs the z scanning mechanism to advance or withdraw the observational field of confocal endomicroscope 14. Images can be taken from a home position near a cover glass window of probe 36 (i.e. at the surface of the site) and advanced into the tissue in steps of approximately 4 μm to a maximum depth, typically of approximately 250 μm. The z depth is controlled either by a user operable control or automated z stack control (not shown). The user operable control may comprise a depth footswitch additionally provided on footswitch 18, or other user operable control, and typically is operable to advance or withdraw the observational field one step at a time.

The automated z stack control, when activated, controls confocal endomicroscope 14 to automatically collect a set of images taken at successively greater depth, advancing the observational field with the z scanning mechanism before each succeeding image. The resulting set of images are termed a ‘z stack’, as they comprise images collected at progressively greater depths into the tissue from the surface to approximately 250 μm (or over a smaller range within this maximum range). The image data constituting the z stack is, as described above, associated with image data collected at essentially the same time with operating microscope 12.

Furthermore, data indicative of the instant depth setting is associated with the image data collected at that respective depth, whether a single image or a z stack is collected. The depth setting may be expressed, for example, either as the number of steps from the home position or—in the z stack case—as the number of steps (forward or back) from the position of the first image in the stack.

In use, laser light from source 42 is transmitted by first optical fibre 50 to optical coupler 44; a first portion of the light is coupled into second optical fibre 52 and transmitted to probe 36. A second portion of the light is coupled into third optical fibre 54 and transmitted to power monitor 46. Probe 36 is adapted to be manipulated manually and placed against the site to be imaged confocally. Before or during such imagining, the power deposited onto the sample can be monitored with power monitor 46 and the known ratio between the power coupled by optical coupler 44 into second fibre 52 and that into third fibre 54. Light returned confocally by the site and collected by probe 36 is transmitted back to optical coupler 44 and a portion of that return light is then coupled into fourth or return optical fibre 56 and transmitted to detection unit 48. An image can then be constructed from the light detected by detection unit 48 and the aforementioned scanning signal, as the latter allows the origin within the sample of the return light to be ascertained.

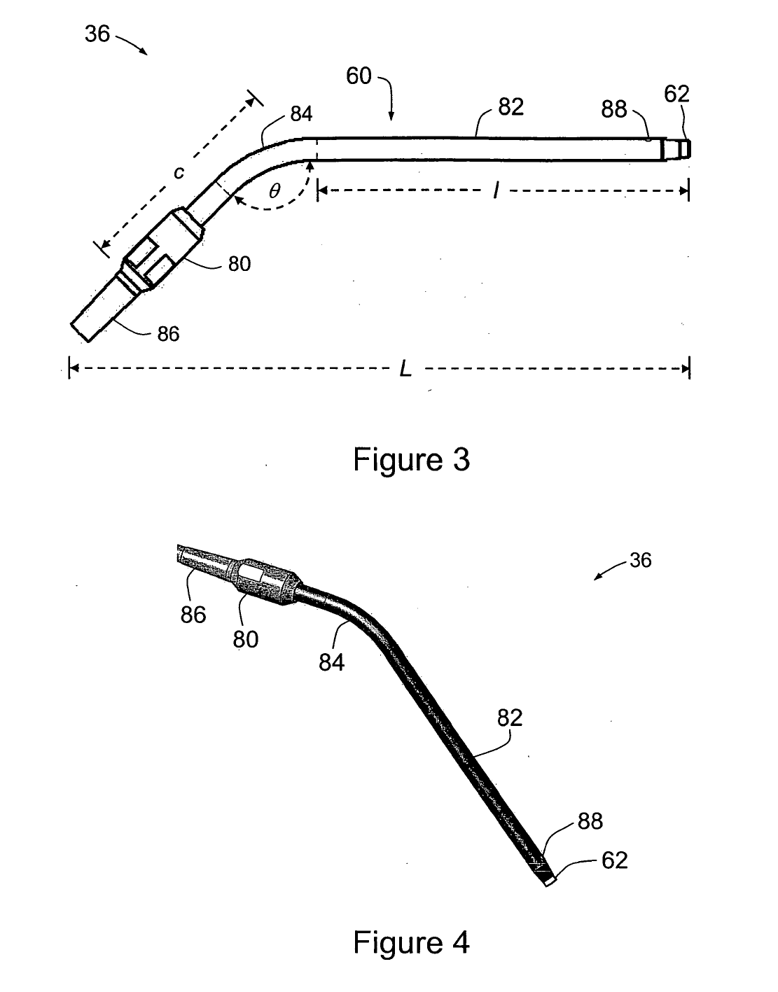

All the optical fibres 50, 52, 54, 56 are single moded at the wavelength of laser source 42, though in some embodiments few- or multi-moded fibre may be used for fourth optical fiber 56. Probe 36 is shown in greater detail in FIGS. 3 and 4, and comprises a rigid steel housing 60 with a distal tip 62 adapted to be placed gently into contact with the site. Housing 60 houses the terminal portion of second optical fibre 52, the scanning mechanism for scanning the exit tip of second optical fibre 52, and an optical train for receiving the scanned light from the exit tip of second optical fibre 52 and focussing it onto or into the site.

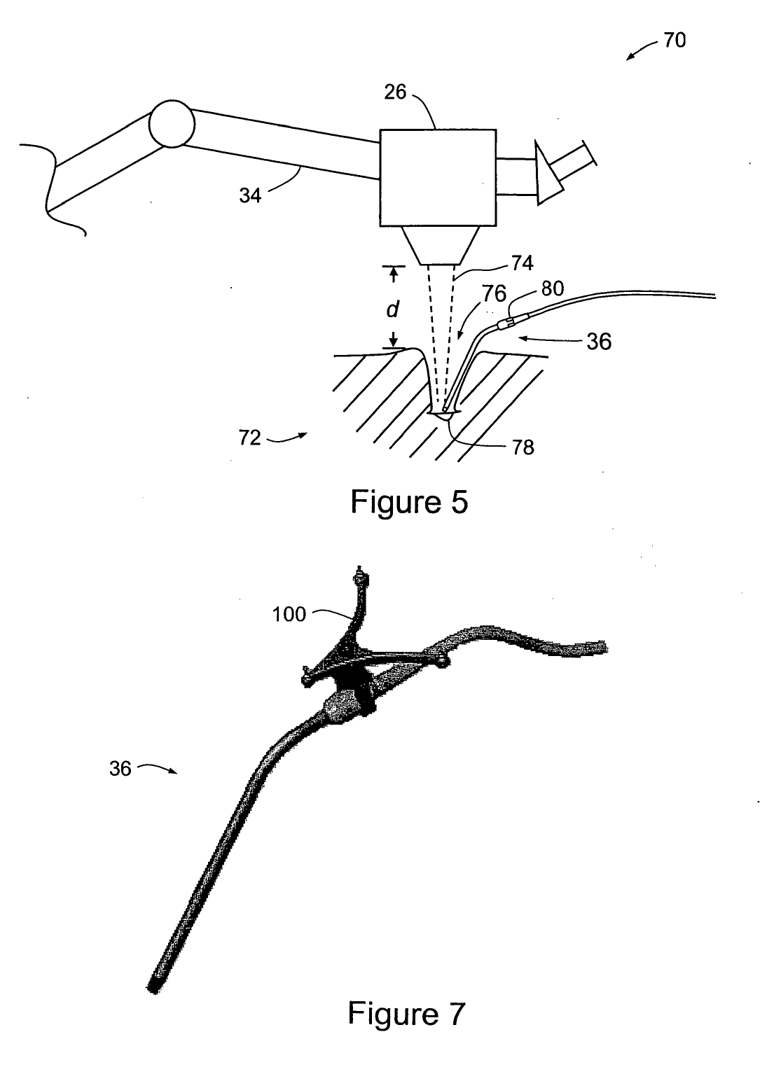

As illustrated schematically at 70 in FIG. 5, confocal endomicroscope 14 is used with operating microscope 12. In use, optical head 26 of operating microscope 12 is supported by arm 34 above a subject 72, and defines an optical corridor 74 into an access corridor 76 created in the subject 72 to provide access to the site 78 under examination. Probe 36, once in position against site 78, can be viewed with operating microscope 12.

Probe 36 is adapted to allow easy fine control of its distal tip 62 by manual manipulation of a proximal portion 80 while distal tip 62 is viewed by operating microscope 12, without probe 36 significantly obstructing optical corridor 74. Probe 36 is thus adapted to be supported comfortably by the operator for accessing site 78 through access corridor 76, and—referring to FIG. 3—has an insertable and essentially straight distal insertion portion 82 with a length of 75 to 205 mm (and, in the illustrated embodiment, approximately 110 mm) and an outside diameter of approximately 6.6 mm. Proximal portion 80 of probe 36 and insertion portion 82 are coupled by a curved portion 84, which introduces approximately a 45° bend between those two portions, so that the angle 0 between proximal portion 80 and insertion portion 82 is approximately 135°. Curved portion 84 allows distal tip 62 of probe 36 to be placed at site 78 with manually manipulated proximal portion 80 held just outside access corridor 76, without proximal portion 80 being in optical corridor 74. Curved portion 84 thus allows the user to have a line of sight through operating microscope 12 along insertion portion 82 of probe 36 that is unobstructed by the user's hands.

In use, insertion of probe 36 into access corridor 76 is accomplished while operating microscope 12 is in place over access corridor 76 and, therefore, probe 36 is dimensioned to fit within the available working distances. For example, for a operating microscope 12 set at a 500 mm working distance and arranged to focus on the deepest structures in an access corridor 76 of 200 mm depth, probe 36 should have a minimum reach of just over 200 mm (and, in practice, no less than 205 mm), provided by insertion portion 82. However, this leaves an access working distance (i.e. between subject 72 and operating microscope 12) d of only 300 mm. Hence, insertion portion 82 (of ≧205 mm), curved portion 84, proximal portion 80 and cable relief 86 should preferably be accommodated by this 300 mm, that is, have a “working length” (i.e. length in a direction parallel to insertion portion 82) of 300 mm. This defines the longest probe dimensions generally usable in this scenario.

In applications where higher magnifications of operating microscope 12 are employed, probe 36 should accommodate shorter working distances. This may involve working at a distance of 200 mm from site 78, with site 78 up to 70 mm deep. In this situation the minimum length of insertion portion 82 would be 75 mm and the total length of probe 36 less than 125 mm to allow probe 36 to be located in the working distance of 125 mm between the subject 72 and operating microscope 12.

Thus the dimensions of probe 36 comprise or depend on the following:

1) insertion portion 82: 75 mm to 205 mm;

2) working length L measured in direction of insertion portion 82: 125 mm to 300 mm;

3) handheld, proximal portion 80, is adapted to sit at a comfortable angle for the position of the user's hand (extending from the bridge between the thumb and index finger to the tips of thumb and index finger);

4) angle θ provided by curved portion 58: between 120° and 150° (and preferably between 130° and 140°, and in this embodiment approximately 135°) 30 between insertion portion 56 and handheld, proximal portion 54;

5) the combined length c of proximal portion 80 and the outer surface of curved portion 84 (together being that part of probe 36 likely to be manipulated by the user during use), in a direction parallel with proximal portion 80, should not be less than the length required for the user to grip probe 36 along this combined length with a minimal number of fingers, while leaving a clear line of sight along the insertion portion 82; this minimum length is estimated to be about 59 mm;

6) combined length c depends on the balance of probe 36 and the available working space: probe 36 should not be unduly heavy in its balance point in respect to the bend; it is estimated that combined length c should not be greater than 75% of the length of the insertion portion 82.

In addition, probe 36 is provided with orientation marking on insertion portion 82, close to distal tip 62, to allow orientation of the ultimate image relative to the field visualised by operating microscope 12. The orientation marking, in the present embodiment, comprises a dot 88 close to distal tip 62, representing “up” in the microscopic field. In other embodiments, however, the orientation marking comprises:

1) a plurality of visually distinguishable dots distributed around insertion portion 82;

2) axially oriented stripes indicating each quadrant (“north/south/east/west” markings);

3) nearly radial markings oriented at an angle to the axis of the scope with the angle being different in different quadrants so that observation from any side enables recognition of which side is being viewed;

4) colour coded markings (such as a plurality of dots, stripes or radial markings) to enhance the differences between different quadrants.

The orientation marking may also comprise any combination of these that serves to allow the identification of the orientation of probe 36.

Confocal endomicroscope 14 orients its output of images collected with probe 36 to correspond to the normal field of view of operating microscope 12, by aligning the “up” direction in that field of view (i.e. typically away from the user) and the top of an image collected with probe 36 when probe 36 is held in a relaxed, neutral manner. Hence, “up” in the confocal image is oriented so that advancing the arm in the direction of the user's forearm with straight wrist will move probe 36 “up” relative to the image. Swinging the arm right from the elbow with straight wrist would move probe 36 right relative to the displayed image, etc.

The optical path for the left and right eye through operating microscope 12 defines a coordinate system for up/down/left/right orientation of the user. The integrated camera of operating microscope 12 can thus be used to measure the outer orientation of probe 36 according to this coordinate system. The orientation of an image generated by confocal endomicroscope 14 can then be transformed to be correctly oriented to the coordinate system of operating microscope 12. This can be done by rotating the endoscopic image, so that up/down/right/left directions coincide with the coordinate system of operating microscope 12. Alternatively, the image orientation of the endoscopic image can be adjusted to the coordinate system of the microscope by transforming the input signals for the scanning mechanism of confocal endomicroscope 14, that is, by rotating the two axes of the scanning mechanism.

In use, therefore, probe 36 of system 10 is positioned by the operator (such as a microsurgeon or neurosurgeon) at various points on the site 78, to collect images for use classifying that the site. This classification may indicate that the site should be resected, biopsied, earmarked for future surveillance, or documented for future reference.

For such classifications to be useful in surgery, surveillance or diagnosis, the anatomical context for the observation is usually important. Knowing the anatomical context of where a microscopic observation was made with confocal endoscope 14, and the association of the interpretation of the microscopic image with that site is thus useful. However, many sites may be imaged in rapid succession a procedure, making manual documentation difficult or impractical.

Further, as probe 36 is positioned by the operator at various points on the site, the microscopic images at each location collected with confocal endoscope 14 may be used to classify that position accordingly. This may include, for example, deciding if the tissue should be resected or left in the patient. As probe 36 is moved across the site, whether continuously or from point to point, such classifications of the site may serve as a map of the locations of various tissue types or margins for resection.

Thus, as a procedure is performed under the high magnification visualization provided by operating microscope 12, the manipulation and positioning of probe 36 is viewed continuously by confocal endomicroscope 14. The view of operating microscope 12 is captured in still or synchronised video format each time an microscope image or short image sequence is collected with confocal endomicro scope 14, to document the location of probe 36 at the time of imaging. The result is the production of a useful data entity associating microscopic observation from confocal endomicroscope 14 with lower magnification view from operating microscope 12 for anatomical context.

System 10 allows additional information to be added as an annotation to the image data stored on computer 16, such as image interpretation, tissue classification or other observations of the surgeon that make the image data more usable. This information could be partially captured at the time of collection and partly in review or post processing, and is added with a data insertion and editing module of image viewing software (now shown) of computer 16. Data insertion and editing module can be operated to capture any desired form of data, including text typed into computer 16, voice inputted with a microphone ultimately coupled to computer 16, indicated by means of an array of footswitches or buttons, selected from a software menu, or otherwise.

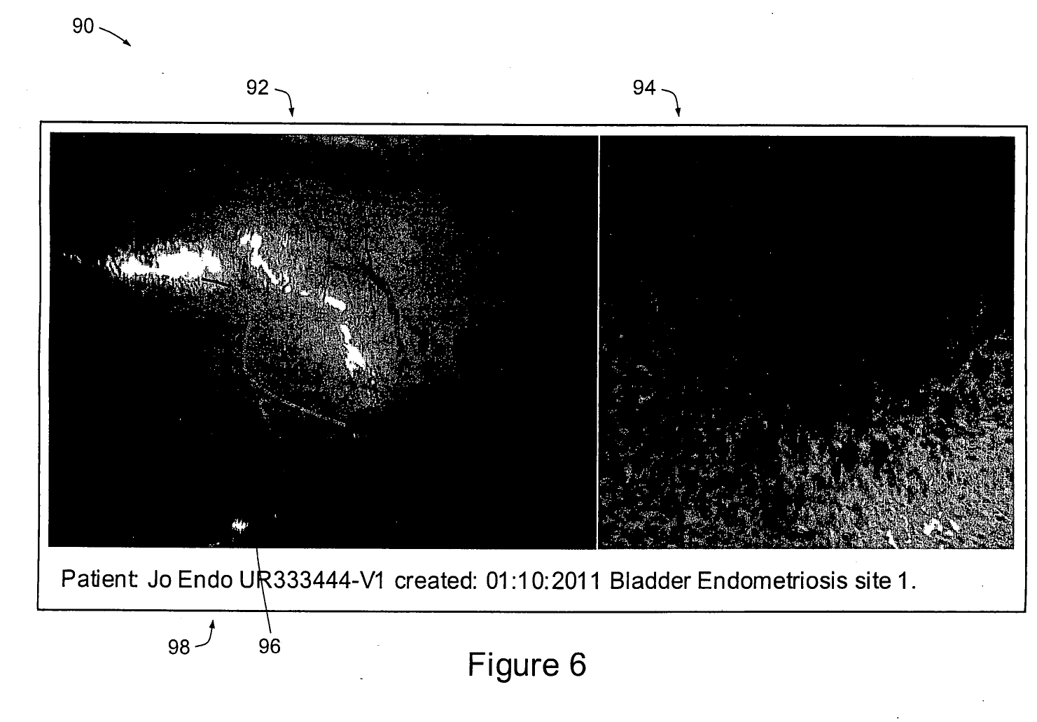

FIG. 6 is an exemplary pair 90 of associated images collected with system 10 of this embodiment. The left register of FIG. 6 is a macroscopic view 92 collected with operating microscope 12, while the right register of FIG. 6 is a microscopic view 94 collected at the same time with confocal endomicroscope 14. Distal tip 62 of probe 36 is visible in macroscopic view 92, so the location of that observational field of microscopic view 94 is apparent. The path of probe 36, deduced from multiple pairs of associated images, can be displayed if desired and, in this example, is indicated by curved path 96. Meta-data 98 indicating the date of image collection, the identity of the subject and nature of the site, is stored with the image data and displayed beneath images 92, 94.

Optionally the localization of multiple microscopic images can be displayed within the macroscopic image 92 in the case of a video stream or a sequence of images. In this case respective microscopic images 94 may either be displayed as a mosaic comprising a plurality of distinct images collected at different locations of probe 36 or a sequence of images corresponding to the actual measurement position in macroscopic image 92.

In another embodiment, system 10 includes a surgical navigation system (not shown). Navigation systems are used in neurosurgery, for example, to provide guidance to surgeons as to the position of surgical tools and to relate that information to other maps based on prior diagnostic imaging, such as CT or MRI scans. According to this embodiment, such a navigation system is instead employed to generate a map or a set of maps intra-operatively by using probe 36 in combination with the navigation system.

In this embodiment, probe 36 is provided with a mechanical reference for the precision mounting of a tracking device or navigation beacon to be tracked in 3D space by the surgical navigation system. The mechanical reference is adapted to facilitate repeatable and precise re-attachment of the tracking device or navigation beacon to probe 36 without needing recalibrating of its position.

FIG. 7 is a view of probe 36 fitted with a navigation beacon 100 for a Medtronic brand surgical navigation system according to this embodiment. The navigation system capable of tracking and recording in real time the position of the tip 62 of endomicroscope probe 36 and therefore the microscopic region being imaged by endomicroscope 14.

The position of probe 36 ascertained with the navigation system can also be correlated with the subject by using the approach described above. That is, at the same moment, operating microscope 12 can be used to collect a macroscopic image of the site and confocal endomicroscope 14 a highmagnification image, while the navigation system outputs the 3D spatial position of probe 36. This is done one or more times, from which the 3D coordinates of the site can be ascertained, and into which subsequent 3D spatial positions of probe 36 can be mapped or displayed.

Furthermore, the navigation system is optionally operable also to track operating microscope 12 or its field of view and output location data indicative thereof. In this embodiment, this location data indicative of the location of probe 36 and that indicative of the location of operating microscope 12 is associated with the image data from confocal endomicroscope 14, so that the location of the image collected with confocal endomicroscope 14 can be shown at their correct positions, added to the surgical field or an image made by operating microscope 12, by means of a data injection module of operating microscope 12.

According to this embodiment, computer 16 is adapted to display—upon demand—a map of those portions of the site imaged with operating microscope 12 and classified from images collected with confocal endoscope 14 to have any of one or more particular tissue classifications. For example, the operator may wish to display the imaged locations classified as “definite tumour” in one colour, and those classified as “normal” in another colour, with those classified as “suspicious for infiltration” in yet another colour. These locations may be displayed on the monitor of computer 16 either as scatter points, or recorded in sequence and displayed with a “join the dots” algorithm (such as is shown at 96 in FIG. 6) to show the path of a believed margin. These points can optionally be displayed on a data injection display of operating microscope 12 or overlayed onto the viewed surgical field.

Also according to this embodiment, system 10 includes a mechanism for relating the position of surgical tools (also ascertained with the surgical navigation system) to the map or maps discussed above and generated with system 10. In addition, computer 16 is programmed with an algorithm adapted to identify tissue properties apparent in the high resolution images made with confocal endomicroscope 14.

Preoperatively defined targets (such as points or areas of interest) in the subject's anatomical dataset (ascertained by, for example, NMR or CT imagery) are accessible by intra-operative guidance of probe 36 to these targets.

In those embodiments of system 10 that do not include a surgical navigation system, an image collected with operating microscope 12 and a highmagnification image collected with confocal endomicroscope 14 may alternatively be correlated by determining a 3D map of the site from stereo images collected with a stereo camera provided in operating microscope 12, ascertaining the positions of endomicroscope tip 62 from the images, storing the positions of endomicroscope tip 62 as a 3D surface dataset, and storing this dataset with the image data.

Modifications within the scope of the invention may be readily effected by those skilled in the art. It is to be understood, therefore, that this invention is not limited to the particular embodiments described by way of example hereinabove.

In the claims that follow and in the preceding description of the invention, except where the context requires otherwise owing to express language or necessary implication, the word “comprise” or variations such as “comprises” or “comprising” is used in an inclusive sense, i.e. to specify the presence of the stated features but not to preclude the presence or addition of further features in various embodiments of the invention. Further, any reference herein to prior art is not intended to imply that such prior art forms or formed a part of the common general knowledge.

1. An imaging system for imaging a surgical site, comprising:
a macroscopic visualization system for imaging the site and generating first image data; and
an imaging apparatus with a probe for locating at the site to define an observational field, the imaging apparatus being adapted to image the observational field and generate second image data;
wherein the system is operable to control the macroscopic visualization system and the imaging apparatus to image the site and the observational field respectively at substantially the same time, and to associate the first image data and the second image data.

2. A system as claimed in claim 1, wherein the imaging apparatus is a microscopic imaging apparatus.

3. A system as claimed in claim 1, including a user operable image collection control adapted to prompt the system to control the macroscopic visualization system and the imaging apparatus to image the site and the observational field respectively.

4. A system as claimed in claim 1, wherein the first image data comprises 3D image data, indicative of a stereo image collected with the macroscopic visualization system.

5. A system as claimed in claim 4, adapted to determine a 3D map of the site from a plurality of images collected with the macroscopic visualization system by storing positions of the probe ascertained from the images as a 3D surface dataset.

6. A system as claimed in claim 1, including a data store adapted to receive the first image data and the second image.

7. A system as claimed in claim 1, including a data output for outputting the first image data and the second image data.

8. A system as claimed in claim 1, configured to tag the first image data with first imaging time data indicative of a time at which the macroscopic visualization system imaged the site, and to tag the second image data with second imaging time data indicative of a time at which the imaging apparatus imaged the observational field.

9. A system as claimed in claim 1, configured to generate a single data file comprising the first image data and the second image data.

10. A system as claimed in claim 1, configured to output the first image data and the second image data at substantially the same time or sequentially.

11. A system as claimed in claim 1, comprising a navigation system, controllable to output a position of the probe.

12. A system as claimed in claim 11, wherein the navigation system is configured to output the position of the probe when the imaging apparatus is controlled to image the observational field.

13. A system as claimed in claim 1, wherein the probe comprises a tip adapted to be located at the site to define an observational field, and to collect a return signal.

14. A system as claimed in claim 13, wherein the probe is an endoscopic probe.

15. A system as claimed in claim 14, wherein the probe is a confocal endoscopic probe.

16. A system as claimed in claim 13, wherein the probe is a neurological probe, an ENT probe, an ultrasound, an OCT probe or a CARS probe.

17. A system as claimed in claim 13, wherein the probe has an orientation marking that allows identification of an orientation of the probe.

18. A system as claimed in claim 17, wherein the orientation marking comprises one or more dots, strips, radial markings or near radial markings.

19. A system as claimed in claim 17, wherein the orientation marking comprises a plurality of portions of different colours.

20. A system as claimed in claim 13, wherein the probe has a manually manipulable proximal portion and a straight distal portion with a distal tip for locating at a site, for emitting light to illuminate an observational field and for collecting return light therefrom, wherein the straight portion has a length of between 75 mm to 205 mm, and the probe has a working length of between 125 mm to 300 mm.

21. A system as claimed in claim 20, wherein the probe has a curved portion between the proximal portion and the distal portion, the curved portion providing an angle between the proximal portion and the distal portion of between 120° and 150°.

22. A system as claimed in claim 1, comprising a z stack control operable to control the imaging apparatus to collect a set of images at successive depths, wherein the second image data is indicative of the set of images.

23. A system as claimed in claim 1, wherein the system is configured to associate depth data indicative of the depth of an image collected with the imaging apparatus with the second image data.

24. A system as claimed in claim 1, comprising a computing device provided with software adapted to identify tissue properties apparent in images made with the imaging apparatus.

25. A system as claimed in claim 1, wherein the imaging apparatus comprises an endomicroscope.

26. An imaging method for imaging a surgical site, comprising:
imaging the site with a macroscopic visualization system and generating first image data;
imaging at substantially the same time an observational field with an imaging apparatus and generating second image data, the imaging apparatus having a probe for defining the observational field to be imaged thereby; and
associating the first image data and the second image data.

27. A method as claims in claim 26, comprising controlling an imaging system comprising the macroscopic visualization system and the imaging apparatus to image the site and the observational field respectively at substantially the same time.

28. An imaging system for imaging a surgical site, comprising:
an imaging apparatus with a probe for locating at a site to define an observational field, the imaging apparatus being adapted to make an image of the observational field and generate image data indicative thereof; and
a locating mechanism for locating the probe and generating location data indicative thereof;
wherein the system is operable to control the imaging apparatus to make an image of the observational field and the locating mechanism to locate the probe at substantially the same time, and to associate the image data and the location data.

29. A system as claimed in claim 28, wherein the locating mechanism comprises a macroscopic visualization system for making an image of the site and generating site image data indicative thereof, wherein the location data comprises the site image data.

30. A system as claimed in claim 28, wherein the locating mechanism comprises a navigation system for locating the probe and generating the location data.

31. A system as claimed in claim 29, wherein the system further comprises a macroscopic visualization system for making an image of the site and generating site image data indicative thereof, and the system is operable to control the macroscopic visualization system to make an image of the site, the imaging apparatus to make an image of the observational field and the navigation system to locate the probe at substantially the same time, the system being configured to associate the site image data, the image data and the location data.

32. A system as claimed in claim 29, wherein the navigation system is operable to locate the macroscopic visualization system or a field of view thereof and generate location data indicative of a location of the macroscopic visualization system or the field of view.

33. An imaging system for imaging a surgical site, comprising:
a macroscopic visualization system for viewing the site and making an image of the site;
an imaging apparatus with a probe for locating at the site to define an observational field, the imaging apparatus being adapted to image the observational field and generate image data; and
a navigation system for tracking a location of the macroscopic visualization system and a location of the probe and generating respective location data indicative thereof;
wherein the system is operable to control the imaging apparatus to make an image of the observational field and the navigation system to locate the macroscopic visualization system and the probe at substantially the same time, the system being configured to associate the image data and the location data.

34. A system as claimed in claim 33, operable to use the location data to indicate the probe location in a field of view of the macroscopic visualization system or in an image made with the macroscopic visualization system.
